(19) United States
(12) Patent Application Publication
(10) Pub. No.: US 2005/0261572 A1
(43) Pub. Date: Nov. 24, 2005

(54) Limb stabilizer for ultra sound detector

(76) Inventors: Vasilis Constantinos Babaliaros, Altanta, GA (US); William Sherman Akin, Tucker, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/850,099

(22) Filed: May 20, 2004

(51) Int. Cl. A61B 5/05

(52) U.S. Cl. 600/415

(57) ABSTRACT

The measuring device includes a cradle (12) for receiving the forearm (30) of a patient. A flexible tether (40) is suspended over the forearm, and an instrument holder (64) is mounted to the lower, distal free end of the flexible tether. The instrument (73) is strapped inside the instrument holder and the technician places the instrument adjacent the desired position of the forearm, and locks the flexible tether in a static position.

### FIELD OF THE INVENTION

This invention concerns a method and apparatus for applying an instrument to a limb of a patient while stabilizing both the limb and the instrument so that the instrument can detect the condition of the limb, such as the forearm.

### BACKGROUND OF THE INVENTION

There are several ways to predict whether people will develop or already have heart disease. If a person does have heart disease, there are several risk factors that we can follow in order to prevent them from having further events.

Classically, there are some things about the body that the patient can control, such as smoking, diet and physical activities; some things we can treat, such as high blood pressure, high cholesterol, and diabetes; and some things we cannot do anything about, such as family history.

Recently there has been a test that has been developed called Flow Mediated Dilation, or FMD, that analyzes the body. Specifically, this test measures the dilating ability of a patient's blood vessels to see how healthy he or she is. Many people believe that this test will be an even stronger predictor of whether you develop heart disease and an even stronger predictor of how bad you will do once you've developed heart disease compared with traditional risk factors.

The basic way the test is performed is simple. It uses ultra sound. Ultra sound can image any vessel in the human body. The brachial artery and the radial artery in the forearm are two vessels that have been traditionally used to measure FMD. Measurement of FMD of the arteries in the forearm is a surrogate test for the health of other blood vessels in the body. This assumption is logical as the whole body is exposed to most of the same conditions.

The way the Flow Mediated Dilation test is performed on the forearm is by placing a small cuff around the wrist, forearm or upper arm (the test has been done using all three locations). The cuff is inflated for five minutes, thus preventing blood flow distally. When the cuff is released, there is a sudden rush of blood to the distal arm. This increase of flow will cause the lining of the blood vessel to produce a molecule called nitric oxide (NO). In a healthy individual, the release of NO will cause the vessel to dilate approximately 5-10%. In an individual with unhealthy blood vessels, the dilation will be less than 5%. Ultrasound is used to image the vessel diameter at baseline, during cuff inflation and after cuff release. Percent dilation is measured as a change of vessel diameter after the cuff is released compared with vessel diameter at baseline.

When conducting the test, two technicians are normally used—one to image the arteries with the ultrasound probe and one to run the other parts of the test that do not involve imaging. The technician in charge of imaging holds the ultra sound probe against the patient's forearm. The probe is about the size of a hand-held tape recorder or smaller (various sizes). The other technician is in charge of the cuff inflation and computer during the test. The information from the probe is fed to a computer and the image of the artery is projected on a monitor screen that is watched by the technicians to make sure the test is being performed properly. The technicians can see the artery dilate with the unaided eye, however, and computer software is often used to accurately measure the diameter of the artery, from edge to edge, as it dilates.

It is difficult to perform FMD of the blood vessels of the forearm because, inherently, these vessels are small in diameter (2-5 mm inner diameter). Any motion between the artery and the ultra sound detector can significantly change the results of the test. Normally, the technician has to stand at the test site for 10-15 minutes holding the ultrasound probe in one position against the patient's forearm. If either the patient or the technicians should move, as to cough or sneeze, the test is often ruined. A very experienced technician is required to perform the ultra sound test and the patient must understand the test and attempt to remain motionless during its entirety.

Thus, there is a need for an apparatus that can stabilize both the patient's arm and the ultrasound detector in order to produce reliable and consistent test results. Not only can such a device improve the accuracy and precision of the test, but it can also reduce the number of technicians needed to conduct the test. This device could be used for other testing besides FMD that requires continuous and accurate imaging of a structure by ultrasound.

### SUMMARY OF THE INVENTION

Briefly described, the present invention comprises a method and apparatus for applying an instrument to a limb of a patient, such as to the forearm of the human body, for the purpose of detecting a condition of the patient, or otherwise treating, testing, or analyzing the patient's limb and other body systems.

A preferred embodiment of the apparatus includes a cradle for receiving the forearm or other limb of the human body, or other animal species, for the purpose of stabilizing the forearm during the test. A tether support, such as a stanchion, is mounted at its proximal end to the cradle structure, so that the tether support extends away from the cradle, preferably in an upward direction over the cradle. In the preferred embodiment, the upper or distal end portion of the stanchion terminates in a horizontally oriented platform. A flexible tether has its upper proximal end mounted to the support platform, and its lower distal free end extends back toward the cradle, and an instrument holder is mounted on the free end of the tether. An instrument, such as a transducer of an ultrasound probe is supported by the holder.

In the preferred embodiment, the tether is flexible and includes a series of modules that are movable with respect to one another, with the modules being connected together by a tensioning cable. When there is slack in the tensioning cable, the modules can move with respect to one another and the distal end of the tether that supports the instrument is free and can be moved about the cradle and the forearm received in the cradle so as to place the instrument in the most appropriate location, usually at the artery that is to be tested. Once in position, the tether can be stabilized by applying tension to the cable, drawing the modules together in static relationships with one another. This causes the tether to become rigid and substantially immovable. The instrument is firmly held against the forearm of the patient.

A preferred embodiment of the invention also may include other features, such as the holder that mounts the instrument to the distal end of the tether is of clam-shaped configuration so that the instrument can be received in the holder with the holder substantially surrounding the instrument for protection and to avoid inadvertent removal of the instrument from the holder.

In the disclosed embodiment, the cradle that supports the forearm of the patient includes a pair of upwardly facing U-shaped receptacles, one for the hand and wrist area and the other for the portion of the forearm adjacent the elbow. These U-shaped receivers are movably mounted to the base of the cradle so that they can be adjusted for different length forearms. In addition, the U-shaped receivers of the cradle can be adjusted for placing the forearm closer or farther away from the stanchion, as may be desired for accurate placement of the instrument at the forearm.

Another feature of the preferred embodiment is that the flexible tether can be rotated along its longitudinal axis so as to manipulate the holder and the instrument held thereby, providing flexibility with respect to the accurate placement of the instrument at the proper location of the forearm, at the artery of the forearm.

A feature of the invention is that the cradle stabilizes the forearm in a preferred position and tends to inhibit any motion of the forearm, in any direction, by longitudinal, lateral or tilting movement of the forearm. This can be enhanced by the inclusion of padding about the forearm, between the forearm and the U-shaped receivers of the cradle, and if necessary, an adjustable length strap or adhesive tape that tends to confine the forearm in the cradle. This can be used in situations where some patients do not have positive control of their limbs.

Typically, the forearm and the flexible tether will be oriented so that the artery faces upwardly and the tether applies the instrument to the upper facing surface of the forearm, so that instrument and the holder as applied by the tether also function as a means for holding the forearm in the cradle, with the patient realizing that any upward movement of the forearm would be resisted by the instrument.

The use of the apparatus is in full sight of the patient so that the patient will understand that any relative movement between the forearm and the instrument is under full control of the patient, not including the technician that applies the instrument to the patient. This effectively eliminates any errors of movement that would otherwise be applied to the instrument by the technician, so that the patient understands that any error in the process is the patient's error.

While the structure of the invention can take many shapes, the method to be performed by the apparatus allows the instrument to be supported by a universally movable distal end of the tether for placement in contact with the forearm or other limb, at the artery of the forearm, etc., in such a manner that complete stability of the limb is achieved, without the need of the technician during the testing procedure, with the instrument steadily applied to the correct portion of the limb without likelihood of disrupting the test.

### BRIEF DESCRIPTION OF THE DRAWINGS

FIG. 1 is a front perspective illustration of the apparatus for holding an instrument.

FIG. 2 is an angled perspective illustration of the apparatus for holding an instrument.

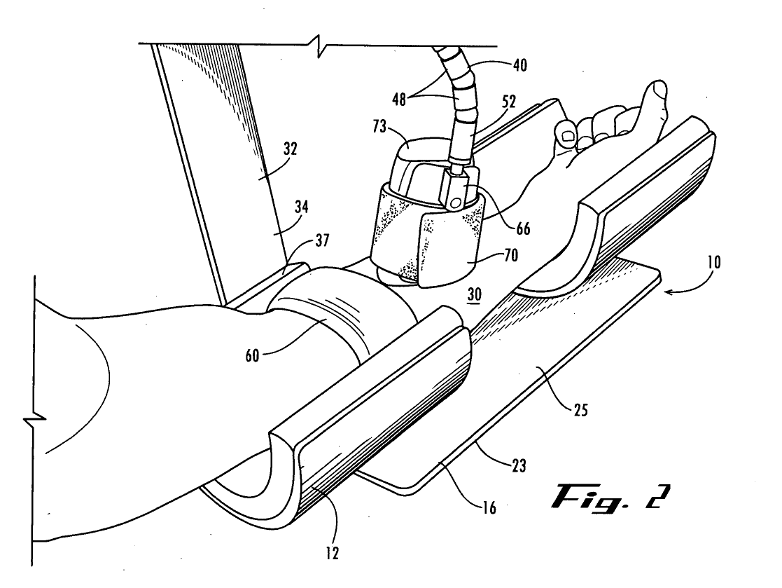

FIG. 3 is a cross sectional view of a portion of the flexible tether.

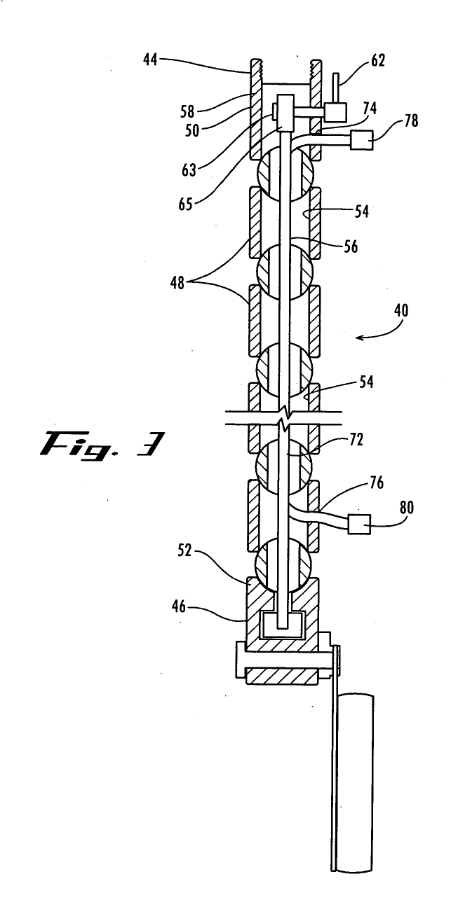

### DETAILED DESCRIPTION OF THE DRAWINGS

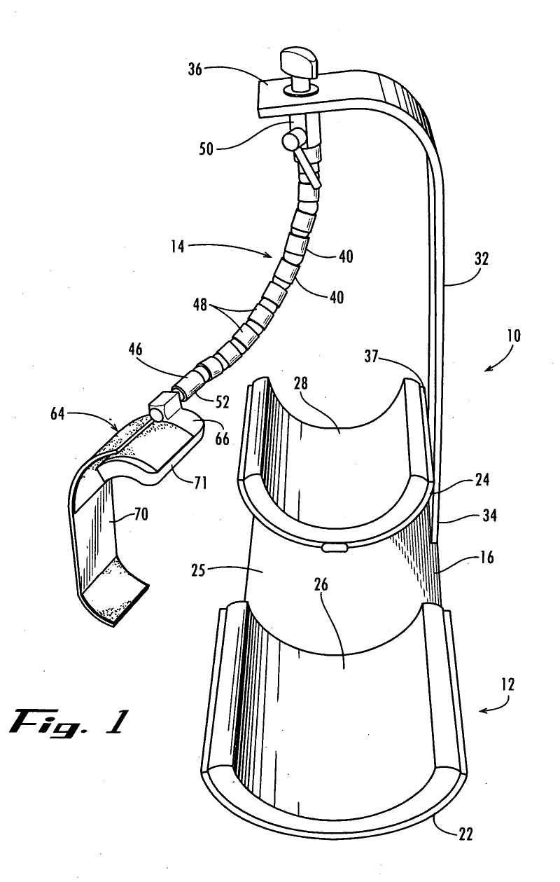

Referring now in more detail to the drawings in which like numerals indicate like parts throughout the several views, FIG. 1 illustrates an embodiment of the artery flow measuring device with its limb stabilizer and its universal positioner for the ultrasound detector, generally indicated by the numeral 10. The apparatus includes a cradle 12 for receiving the limb 30 of a human body, particularly the forearm of a patient, and a universal positioner 14 supported by the cradle for applying an instrument in juxtaposition with an artery of the limb received in the cradle.

The cradle includes a flat base plate 16 that is to rest on a horizontal foundation, such as a table top, on the surface of the mattress of a hospital bed, or substantially any other available and convenient horizontal surface. A pair of U-shaped limb receivers 22 and 24 are mounted on the base plate 16 at opposite ends of the base, leaving a gap 25 between the limb receivers. A resilient pad 23 may be applied to the bottom surface of the base plate 16 to retard movement of the limb stabilizing device on its supporting surface. The size of the open portion of the U-shaped limb receivers can be adjusted with the use of pads, such as pads 26 and 28 in the receivers, for accommodating forearms of different sizes. Also, the pads tend to stabilize the forearm to minimize any movement of the forearms during the tests to be performed by the apparatus. The pads can be pillows filled with soft material, with air, or can be sponge material. Particularly the U-shaped limb receivers 22 and 24 and their pads tend to avoid turning of the forearm, tilting of the forearm, or lifting of either end of the forearm, or sliding the forearm longitudinally.

If desired, adhesive tape or a Velcro strap (not shown) or other type restraining devices can be used to extend about the forearm and either of the U-shaped limb receivers 22 and 24, or pass across the arm, extending through the gap 25 between the U-shaped limb receivers 22 and 24.

The gap 25 between the U-shaped limb receivers allows space for the placement of the instrument holder and its instrument, such as the ultrasound transducer, as well as a blood pressure cuff (not shown) that is inflated without being hindered by the base 16 or the U-shaped limb receivers 22 and 24. The holder of the ultrasound transducer can be moved circumferentially about the forearm without being inhibited by the U-shaped limb receivers 22 and 24.

Universal positioner 14 is supported by the cradle 12 and includes a tether support 32 which, in this embodiment, comprises an upright stanchion having a lower or proximal end 34 rigidly mounted to the base 16. The stanchion can be formed of rigid material, such as plastic, nylon, metal, wood or other materials that provide a stable and strong support for the apparatus. In the embodiment illustrated, the stanchion includes a metal strap welded at 34 to the flat base plate 16 and to an edge 37 of the U-shaped limb receiver 24. The upper or distal end portion of the stanchion is formed in an arc at 39 so that it transitions from a vertical orientation to a horizontal orientation, and a platform 36 is formed on the horizontal upper distal end portion of the stanchion.

The universal positioner 14 further includes a flexible tether 40 of prior art design having an upper proximal end portion 44 and a lower distal free end portion 46. The tether 40 includes a series of modules 48 positioned in abutting relationship with one another, with the proximal end 44 of the tether terminating in upper mounting tube 50, and the lower distal end portion 46 of the tether terminating in lower mounting tube 52. The upper and lower mounting tubes 50 and 52, and each of the modules 48, define aligned central passages 54, and a tension cable 56 passes through the passages, with the tension cable being fastened at one end to the lower mounting tube 52. The upper end of the tension cable includes a lever 62 that is rotatably mounted in the upper mounting tube 50 to apply tension to the tension cable or to relax the tension cable. The lever 62 extends out from the upper mounting tube 50 to be accessible from the outside of the flexible tether 40 and has a cam 63 mounted internally of the upper mounting tube 50. A cam follower 65 at the upper proximal end of the tension cable 56 moves in response to the movement of lever 62 and cam 65 to apply tension to the tension cable. This rigidifies the tether 40.

Instrument holder 64 is mounted to the lower mounting tube 52 of the flexible tether 40. Instrument holder 64 includes an arcuate “clam shell” plate 66 and a hook and loop elastic fastener belt 70 wrapped thereabout. Padding 71 is applied to the concave surface of the clam shell plate, and an instrument 73 such as an ultrasound transducer is held between the clam shell plate and the hook and loop fastener belt 70, with the strap applying compression to the clam shell plate, the pad, and the instrument 73.

Electrical wire conductors 72 may extend through the central passages 54 of the modules 48, and through openings, such as opening 74 in one of the upper modules 48 and the opening 76 in one of the lowermost modules 48 adjacent the lower mounting tube 52. Plugs 78 and 80 are joined to the ends of the electrical conductors for the purpose of connecting the instrument 73 to a computer (not shown).

### Operation

When the apparatus is to be placed in use, the technician applies an inflatable cuff 60 to the patient and places an instrument 73 such as an ultrasound transducer between the clam shell plate 66 and the belt 70 of the instrument holder 64, and the hook and loop elastic fastener belt 70 is wrapped around the clam shell plate and its ends are secured together. The padding 71 between the concave surface of the clam shell plate and the instrument 73 applies compressive force to the instrument 73 so that the instrument is maintained by friction in position at the end of the tether. The instrument 73 is electrically connected by plugs 78 and 80 to a computer, with the electrical conductors 72 extending from the instrument through the modules 48 of the flexible tether 40.

The patient is instructed to place his or her forearm 30 in the U-shaped limb receivers 22 and 24 of the cradle 12. The gap 25 is maintained between the U-shaped limb receivers and the inflatable cuff 60 on the forearm of the patient may occupy some of the space between the U-shaped receivers. If desired, the U-shaped limb receivers can be adjustably mounted to the base plate 16 (not shown) so that they can be moved longitudinally with respect to the base plate 16, so as to accommodate the inflatable cuff and limbs of different lengths and shapes.

The technician then adjusts the free end of the tether to place the instrument holder 64 with its instrument 73 into contact with the forearm of the patient, with the instrument being carefully placed in the vicinity of the forearm that corresponds to the position of the artery that is to be measured and making sure the instrument is positioned on the distal side of the inflatable cuff. During this operation the lever 62 will have been oriented by the technician as necessary so as to remove the tension from the internal tensioning cable of the flexible tether, so that the tether 40 remains flexible and its lower distal free end can be moved to virtually any position or attitude about the forearm.

Once the technician is satisfied that the instrument 73 is properly positioned, usually by watching the monitor of the computer, the technician then rotates the lever 62 which applies tension to the tension cable. This urges the modules 48 into compressive, frictional contact with one another, so that each module assumes a fixed position with respect to the next adjacent modules, and the net effect is that all of the modules become rigidly connected to one another, thereby rigidifying the tether 40. This causes the instrument 73 to remain in the desired position at the forearm 30 of the patient.

The technician inflates the cuff about the patient's forearm to constrict the blood flow through the patient's forearm.

Since the U-shaped limb receivers 22 and 24 are aligned with one another and with the length of the forearm 30 of the patient, the padding 26, 28 in the limb receivers 22 and 24 tends to firmly stabilize the forearm of the patient. If desired, adhesive tape, a hook and loop belt fastener, or other retention means can be extended over the forearm and adhered or fastened to the base plate 16 of the cradle, to further stabilize the forearm.

It will be noted that the forearm 30 usually is oriented so that its upwardly facing surface bears the artery that is to be measured, and the instrument 73 and its holder 64 are positioned above the forearm. This position of the instrument tends to trap the forearm in the cradle, giving the patient the sensation that the forearm is not to be moved with respect to the instrument or the cradle. Indeed, the technician can apply additional downward force to the instrument holder 64 so that it applies mild force to the upwardly facing surface of the forearm, tending to hold the forearm in place in the cradle.

The apparatus is constructed and configured so that once it has properly received the forearm of the patient and the instrument is applied thereto as described above, the technician is free to operate the testing equipment without providing close attention to the patient. Thus, only one technician is required for performing the entire test on the patient.

After a time delay of, for example 5 minutes, during which the cuff is inflated and constricts the forearm and the instrument makes a reading as displayed on the monitor by the technician, the technician deflates the cuff and the instrument and the computer continue to record the performance of the artery until stability of the artery is detected. Alternatively, the computer can control the deflation of the cuff at the completion of the test.

Although a preferred embodiment of the invention has been disclosed in detail herein, it will be obvious to those skilled in the art that variations and modifications of the disclosed embodiment can be made without departing from the spirit and scope of the invention as set forth in the following claims.

1. Apparatus for holding an instrument juxtaposed the limb of a human body, comprising:
a cradle for receiving the limb of a human body,
a tether support mounted to said cradle and having a distal portion extending away from said cradle,
a tether having a proximal portion mounted to the distal portion of said tether support and a distal portion for extending toward juxtaposition with a limb received in said cradle,
an instrument holder mounted to the distal portion of said tether for supporting the instrument, and
an instrument supported by said instrument holder for placement at the limb received by said cradle.

2. The apparatus for holding an instrument as set forth in claim 1, and further including:
said tether being flexible and including a lock assembly for stabilizing said holder adjacent the limb received in said cradle.

3. The apparatus for holding an instrument as set forth in claim 2, wherein:
said tether is formed of a plurality of modules mounted in series, and
said lock assembly comprises a tensioner for drawing together said plurality of modules into static frictional engagement with one another.

4. Apparatus for holding an instrument juxtaposed a limb of a human body for non-invasive measuring of flow mediated dilation of arteries, comprising:
a cradle for receiving a limb of a human body, said cradle including opposed ends configured to receive and immobilize the ends of the limb,
a holder for receiving an ultrasound transducer,
a universal positioner supported by said cradle for supporting said holder in juxtaposition with an artery of the limb received in said cradle,
said universal positioner including a flexible tether having a proximal end portion supported by said cradle and a distal end portion for supporting said holder,
said flexible tether including tensioning cable and a series of modules mounted on said cable and movable with respect to one another about said cable, and
a tensioning lock configured to apply tension to said cable and urge the modules into frictional locked engagement with one another and rigidify said tether and maintain said holder in a fixed position juxtaposed the limb in said cradle.

5. The apparatus of claim 4, and further including an ultra sound transducer received in said holder.

6. The apparatus of claim 4, wherein
said universal positioner includes a stanchion mounted to said cradle and having an upper end extending upwardly and laterally over said cradle, and
said proximal end portion of said flexible tether connected to said stanchion and said tether is suspended from said stanchion.

7. The limb stabilizer of claim 6, wherein said tensioning lock is positioned at said upper end of said stanchion.

8. The limb stabilizer of claim 4, wherein
said cradle comprises base and a pair of U-shaped limb receivers spaced from each other mounted on said base, and said universal positioner mounted on said base between said U-shaped limb receivers.

9. The limb stabilizer of claim 8, wherein
said base includes a slot and said U-shaped limb receivers are mounted in said slot.

10. The limb stabilizer of claim 4, wherein
said instrument holder is clam shell shaped with modules movable toward each other of grasping the instrument.

11. The limb stabilizer of claim 4, wherein
said modules of said tether define openings there through, and said tensioning cable extends through said openings.

12. The limb stabilizer of claim 4, wherein
said instrument has electrical conductor leads that extend from the instrument along said tether.

13. Apparatus for holding an instrument juxtaposed the arm of a human body for measuring the internal characteristics of the human arm, comprising:
a cradle for receiving a forearm of a human body, said cradle including opposed ends configured to receive and immobilize the elbow and wrist of the forearm,
a holder for receiving an ultrasound transducer,
a transducer received in said holder,
a universal positioner supported by said cradle for supporting said holder in juxtaposition with an artery of the forearm received in said cradle,
said universal positioner including a stanchion mounted to said cradle and having an upper end extending upwardly and laterally over said cradle, a flexible tether having a proximal end portion supported by said stanchion over said cradle and a distal end portion suspended over said cradle and supporting said holder,
said flexible tether including cable and a series of modules mounted on said cable and movable with respect to one another about said cable, and
a tensioning lock configured to urge the modules into frictional locked engagement with one another and rigidify said tether,
so that a fore arm is placed in the cradle, the ultrasound detector is received in said holder, said distal end of said flexible tether is moved to place the ultrasound detector in juxtaposition with an artery of the forearm placed in said cradle, and said tensioning lock shortens the length of said cable to urge said modules of said flexible tether into frictional engagement with one another to hold said ultrasound detector in position with the artery of the arm in said cradle.

14. A method of applying an instrument to a limb of a patient, comprising:
providing a cradle having a longitudinal limb support recess,
providing a tether support having a proximal end portion supported by the cradle and
having a distal end portion positioned generally above the longitudinal limb support recess,
mounting the tether from its proximal end to the distal end portion of the tether support with distal end portion of the tether suspended adjacent the cradle,
supporting an instrument in the distal end of the tether support,
placing a limb of the patient in the cradle with the length of the limb oriented along the longitudinal limb support recess,
adjusting the tether to place the instrument in contact with the limb, and
locking the tether in a static position with the instrument in contact with the limb.

15. The method of claim 14, and further including the step of
strapping the limb of the patient in the cradle.

16. The method of claim 14, and further including the step of
adjusting the position of the tether support with respect to the cradle for changing the position of the instrument.

17. The method of claim 14, wherein said cradle includes a pair of U-shaped limb receivers spaced from each other and defining a gap there between, and the step of adjusting the tether includes placing the instrument against the forearm at a position at or in the gap between the U-shaped limb receivers.

18. Apparatus for holding an instrument adjacent the limb of a human body, comprising:
a cradle for receiving the limb of a human body, said cradle including a pair of U-shaped limb receivers aligned with each other and spaced from each other to form a gap there between,
a tether supported by said cradle having a free end portion for extending toward said gap and into juxtaposition with a limb received in said cradle,
an instrument holder mounted to the distal portion of said tether for supporting the instrument, and
an instrument supported by said instrument holder for placement in said gap in contact with the limb received by said cradle.

19. The apparatus of claim 18, wherein said tether is flexible and includes a lock for rigidifying said tether.
